(19) United States
(12) Patent Application Publication
(10) Pub. No.: US 2024/0399288 A1
(43) Pub. Date: Dec. 5, 2024

(54) TECHNOLOGICAL LINE FOR THE SEPARATION OF METHANE FROM A MIXTURE OF GASES DISCHARGED FROM A COAL MINE AND A METHOD FOR THE SEPARATION OF METHANE FROM A MIXTURE OF GASES DISCHARGED FROM A HARD COAL MINE

(71) Applicant: BIOPOLINEX SPOLKA Z OGRANICZONA ODPOWIEDZIALNOSCIA, Lublin (PL)

(72) Inventors: Dobieslaw Nazimek, Lublin (PL); Andrzej Pryzowicz, Lublin (PL); Krzysztof Pietrzak, Lublin (PL); Stanislaw Tadeusz NAWRAT, Wodzislaw Slaski (PL)

(21) Appl. No.: 18/689,065

(22) PCT Filed: Aug. 30, 2022
(86) PCT No.: PCT/IB2022/058105

§ 371 Date: Mar. 4, 2024

(30) Foreign Application Priority Data
Sep. 2, 2021 (PL) P.438872

(51) Int. Cl. B01D 53/14; B01D 53/18; B01D 53/26; C07C 7/00

(52) U.S. Cl. CPC B01D 53/1425; B01D 53/1406; B01D 53/1431; B01D 53/18; B01D 53/26; C07C 7/005; B01D 2252/20; B01D 2257/7025; B01D 2257/80; B01D 2258/02

(57) ABSTRACT

The technological line for the separation of methane from the mixture of gases discharged in the exhaust shafts from the coal mine has a diffuser (2) installed in the exhaust shaft (1) connected with an installation conduit (3) with a dehydrator (4) and a drying device (5) and a fan (6) and a set of filters (7) coupled with at least one absorber (8) filled with saturated paraffin oil, and the draining installation conduit (9) from the absorber (8) has a paraffin oil dropplet (10) connected to the expansion tank (11) by an installation conduit (11) (12), and the installation conduit (13) connecting the absorber (8) with the expansion tank (12) has a pump (14) coupled with the desorber (15), which is connected to the utility installation with installation conduits (16), the evaporator (15), is also connected with an installation conduit (17) with a pump (18) coupled with a cooler (19), and an eqalizing tank (20), which is connected to the absorber (8) through an installation conduit (21) in a closed system. The line enables the separation of methane from the mixture of gases discharged with the mine ventilation air and its utilization. The invention also relates to a highly efficient process for the removal and utilization of methane as a greenhouse gas from deep hard coal mines, allowing to obtain methane with optimal concentration parameters from the mixture of gases discharged from the underground coal mine through the exhaust shafts.

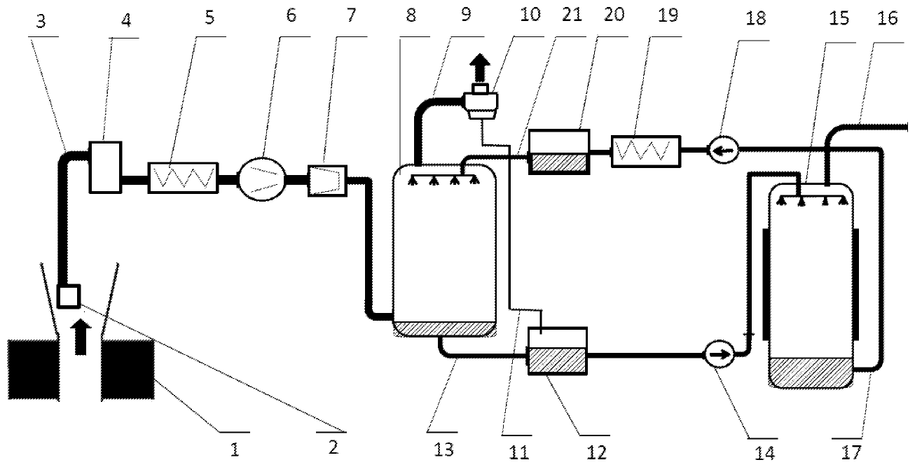

The subject of the invention is a technological line for the separation of methane from a mixture of gases discharged in exhaust shafts from a hard coal mine and a method for the separation of methane from a mixture of gases discharged from a hard coal mine through exhaust shafts.

In mines, the methane accompanying the exploitation of the basic mineral coal, not captured by methane drainage, is mostly released into the ventilation air, forming methane-air mixtures with different methane concentrations. The use of methane from coal seams is very important for: economic reasons, which was reflected in the Geological and Mining Law, which classifies methane from coal seams as basic, ecological minerals, because the emission of methane to the atmosphere contributes to the greenhouse effect.

A major problem in Polish and global mining is the utilization and economic use of methane from mine ventilation air (MWENT). In mines, methane from coal seams is released into the air in the mine during the coal mining process and is diluted to form in the ventilation shafts, as a result of air flow regulation, methane-air mixtures (MWENT) containing from 0.01% to 0.75% of methane per outlets from ventilation shafts (0.75%—the maximum permissible methane content in ventilation shafts specified in the Polish mining safety regulations). Polish mining regulations require that the methane content in the air in ventilation shafts be lower than 0.75%. The basic means of limiting and controlling the methane content in the air in mining excavations is the supply of sufficiently large air streams. Therefore, the content of methane in ventilation shafts ranges from 0.01% to 0.5% and such mixtures with a very low methane content are of little use as energy sources.

For many years, Polish hard coal mines have been gradually developing underground demethanation and economic use of captured methane in heating and energy installations.

In principle, the available technological solutions use three methods of separating methane from other pollutants, mainly nitrogen. These are: the cryogenic method, which uses a relatively large difference in condensation temperatures between the gases—methane and nitrogen, the membrane separation method and the methane absorption method on porous carbon materials.

The methane enrichment method-low-temperature method is used most often, also in Poland.

In the search for alternative methods of storing gaseous fuels, an important role may be played by the use of absorption technology [Czepirski 1989, 2007; Lozano-Castello et al. 2002; Nijkamp et al. 2001; Texier-Mandoki et al. 2004]. Absorptive storage of gaseous fuels is justified in cases where the density of the absorbed gas is sufficiently greater than that of the gas phase to compensate for the reduction in space available for gas due to the presence of the absorbent.

In the early nineties of the twentieth century, the US Department of Energy, as a condition of practical application, determined the value of the storage capacity of methane at the level of 150 volume units of gas per unit volume of the tank under the pressure of 3.5-4.0 MPa. This is equivalent to a gas compressed to a pressure of 14-16 MPa. The above pressure range for absorption storage also has a utilitarian significance. In the absorption system, it is possible to eliminate expensive high-pressure compression systems and replace them with filling the tanks with small two-stage compressors or even directly from gas networks.

Moreover, methods of separation of methane from other gases using hydrate formation are known in the art [WO2018118623, 2016] or by high pressure thermal treatment [CN101485949, 2009, WO9741085, 1996, US2010292524, 2006]. In all the above-mentioned methods of separation of methane from the gas phase, the factor determining their application is the economy of the process. In the case of ventilation air from mines, where the methane concentration cannot exceed 0.75%, these methods are not economically justified.

The methods and systems known so far have not made it possible to obtain high efficiency and economic efficiency, for the separation of methane from the mixture of gases discharged with the mine ventilation air.

The problem was also solved in the invention, where, thanks to the installation and method of separation of methane from the mixture of mine gases, in which a solvent was used, enabling the absorption of methane and thus creating a new compound structure. The process is carried out in a closed system, where the same solvent in the form of paraffin oil is repeatedly used, which, after methane desorption, is returned to the repeated methane absorption process. In addition, the method and technological line for the separation of methane from the mixture of gases discharged in exhaust shafts from a hard coal mine enables the utilization of methane (greenhouse gas) currently discharged into the atmosphere along with the VAM ventilation air from underground methane coal mines, which has a negative impact on the environment. The invention, thanks to conducting the process in a closed system and recycling of the solvent used for the absorption of methane, enables the efficient production of pure methane at relatively low cost of the process compared to the known methods.

The aim of the invention is to develop a highly efficient system and method enabling the discharge and utilization of methane as a greenhouse gas from deep hard coal mines, allowing to obtain a product in the form of methane with optimal concentration parameters up to 99.9% from the mixture of gases discharged from the underground coal mine through exhaust shafts.

The essence of the technological line for the separation of methane and the mixture of gases discharged in exhaust shafts from a hard coal mine, according to the invention, is that in the exhaust shaft there is a diffuser connected by an installation conduit with a dehydrator and drying device, as well as a fan and a set of filters coupled with at least one absorber filled with saturated paraffin oil, and the installation pipe draining from the absorber has a paraffin oil droplet separator connected to the equalizing tank, and the installation conduit connecting the absorber with the equalizing tank has a pump coupled with the desorber, where the separated methane is led through an installation conduit to utility installations, the desorber is equipped with a pump for paraffin oil, coupled with the cooler and the equalizing tank, which is connected through the installation conduit to the absorber in a closed system.

Preferably the line has at least 2 absorbers connected in series or in parallel. Preferably, the absorbers are equipped with Raschig and/or Biatecki rings. Preferably the evaporator is heated to a temperature of at most 75° C.

The technological line according to the invention makes it possible to carry out a highly efficient process of removing and utilizing methane as a greenhouse gas from underground hard coal mines, from the mixture of gases discharged from the underground coal mine through exhaust shafts through the separation of methane from the mixture of mine gases. The installation allows for the methane separation process in a closed system with multiple use of the same solvent.

The essence of the method of separating methane from a mixture of gases discharged from a coal mine through exhaust shafts is that first, water is removed from the gas mixture containing methane, and then the dehydrated mixture is dried to a humidity of at most 65% relative humidity and contacted in the absorber with paraffin oil light containing alkanes from C₁₀-C₁₄ at a temperature not higher than 20° C., and the unabsorbed gases are discharged to the atmosphere, and the paraffin oil saturated with methane is heated in a desorber to a temperature of at least 50° C., preferably 75° C., and the process of methane separation is carried out to a concentration of 99-100%, which is then fed to utility installations, while the paraffin oil remaining after the separation of methane, is returned in a closed system to the repeated methane absorption process, whereby it is first cooled to a temperature not exceeding 20° C.

Preferably, the evaporator is purged with nitrogen or other inert gas prior to the desorption process.

The solution in question allows to obtain a product in the form of methane with an optimal concentration of 99-100% from the mixture of gases discharged from the underground coal mine through the exhaust shafts to the atmosphere. The solution is characterized by high process efficiency and enables the utilization of methane as a greenhouse gas discharged into the atmosphere from underground methane hard coal mines. The invention, thanks to conducting the process in a closed system and recycling of the solvent used for the absorption of methane, enables the efficient production of pure methane at relatively low cost of the process compared to the known methods.

### EXAMPLE OF THE LINE

The subject of the invention is shown in the embodiment in the drawing, which schematically shows the technological line for the separation of methane from the mixture of gases discharged in the exhaust shafts from the coal mine.

In the exhaust shaft 1 of the underground coal mine, a diffuser 2 is installed, connected by an installation conduit 3 to a dehydrator 4 and a drying device 5, as well as a fan 6 and a set of filters 7, which are coupled to an absorber 8 equipped with Biatecki rings increasing the absorption surface. The absorber 8 is filled with saturated paraffin oil, and the draining installation conduit 9 from the absorber 8 has a paraffin oil droplet 10 connected by an installation conduit 11 with the equalizing tank 12, and the conduit 13 connecting the absorber 8 with the equalizing tank 12 has a pump 14 coupled with a desorber which is evaporator 15, which is connected by installation lines 16 to the utility installation, while the evaporator 15 is also connected by installation line 17 to a pump 18 coupled to a cooler 19 and an equalizing tank 20, which is connected to the absorber 8 in a closed system through the installation line 21.

### Example of the Method

First, water was removed from the gas mixture discharged from the coal mine by means of methane-containing exhaust shaft 1 in a dehydrator 4, then the dehydrated mixture was dried in a drying device 5 to 60% relative humidity, and then it was contacted counter-current with light paraffin oil containing alkanes from C₁₀-C₁₄ at a temperature of 19-20° C. in a sprinkler-type absorber 8 with a capacity of not less than 2 m³, and the absorption process was carried out. The unabsorbed gases were discharged to the atmosphere, while the paraffin oil saturated with methane in the amount of 14% was heated in the evaporator to the temperature of 75° C. and the process of methane separation brought to the concentration of 99-100% was carried out, which was then introduced into utility installations. The paraffin oil remaining after the separation of methane is recycled in a closed system to the methane reabsorption process, whereby it is first cooled to 20° C. and the evaporator 15 was purged with nitrogen prior to the desorption process. The absorber 8 was equipped with Biatecki rings increasing the absorption area to a total capacity of 2000 dm³ (2 m³). Moreover, the absorber 8 was equipped with pumps for feeding and receiving the solvent with a capacity of 6 dm³/min, and the evaporator 15 had a capacity of 10 dm³ and was heated to a temperature of 75° C. The installation included a cooler 19 to cool the solvent flowing from the evaporator 15 to a temperature of 20° C. The entire installation was automatically controlled with simultaneous computer notation, changes in the concentration of methane at the inlet/outlet of the absorber 8 and the desorber—evaporator 15, air flow rate and temperatures of the absorber 8, desorber—evaporator 15 and cooler 19. The tests carried out at the air volume velocity of 65.7 m³/min containing methane at a concentration of 0.45% and the solvent feed rate of 4 dm³/min showed that the total efficiency of the installation (absorber and evaporator) under these operating conditions is 84.3%. The stream of air fed to the absorber 8 contained a stream of methane with a space velocity of 30.79 dm³/min, i.e. 1852.74 dm³/h. As a result of desorption in the evaporator 15, a stream of methane of 99.9% purity, on the order of 1,561.859 dm³/h, was obtained, thus prooving the correctness of the assumptions for the construction of the installation. During the process, the solvent used for absorption was fed at a rate of 4 dm³/min, after cooling from the temperature of 75° C. to 20° C., it was re-fed to the absorber 8.

1. Technological line for the separation of methane from the mixture of gases discharged in exhaust shafts from a coal mine, characterized in that it has a diffuser (2) installed in the exhaust shaft (1) connected with an installation pipe (3) with a dehydrator (4) and a drying device (5) and a fan (6) and a set of filters (7) coupled with at least one absorber (8) filled with saturated paraffin oil, and the draining installation pipe (9) from the absorber (8) has a paraffin oil droplet (10) connected with an installation pipe (11) with the equalizing tank (12), and the installation conduit (13) connecting the absorber (8) with the equalizing tank (12) has a pump (14) coupled with the desorber (15), which is connected to the utility installation with installation conduits (16), where the desorber (15) is also connected by an installation conduit (17) with a pump (18) coupled to the cooler (19), and an equalizing tank (20), which is connected through the installation conduit (21) to the absorber (8) in a closed system.

2. The line according to claim 1 characterized in that it preferably has at least two absorbers (8) operating in series or in parallel.

3. The line according to claim 1, characterized in that the absorbers (8) are equipped with Raschig and/or Biatecki rings.

4. A method of separating methane from a mixture of gases discharged from a coal mine through exhaust shafts, characterized in that water is removed from the gas mixture containing methane, and then the dehydrated mixture is dried to a humidity of at most 65% of relative humidity, and contacted in an absorber with light paraffin oil containing alkanes from C₁₀-C₁₄ at a temperature not higher than 20° C. and the absorption process is carried out, and the unabsorbed gases are discharged to the atmosphere, while the paraffin oil saturated with methane is then heated in a desorber to a temperature of at least 50° C. and the process of methane separation is carried out, followed by is discharged into utility installations, while the paraffin oil remaining after the separation of methane is returned to the absorber in a closed system for the repeated methane absorption process, whereby it is first cooled to a temperature not higher than 20° C.

5. Separation method according to claim 4, characterized in that the evaporator is purged with nitrogen or other inert gas prior to the desorption process.

6. The separation method according to claim 4, characterized in that the methane-saturated paraffin oil is heated in a desorber to a temperature of 75° C.
